(19) United States
(12) Patent Application Publication
(10) Pub. No.: US 2010/0121398 A1
(43) Pub. Date: May 13, 2010

(54) IMPLANTABLE MEDICAL DEVICE AND METHOD FOR MONITORING VALVE MOVEMENTS OF A HEART

(75) Inventors: Anders Björling, Solna (SE); Cecilia Tuvstedt, Stockholm (SE); Kenth Nilsson, Akersberga (SE); Kjell Norén, Solna (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St. Jude Medical AB

(21) Appl. No.: 12/597,227

(22) PCT Filed: Apr. 27, 2007
(86) PCT No.: PCT/SE2007/000411

§ 371 Date: Oct. 23, 2009

(51) Int. Cl. A61N 1/368

(52) U.S. Cl. 607/17

(57) ABSTRACT

An implantable medical device for monitoring the movements of the valve planes of the heart to determine at least one hemodynamic measure reflecting a mechanical functioning of a heart of a patient, includes an impedance measuring circuit that measures impedance between at least electrode pairs including at least one electrode placed substantially at the level of the valve plane. The measured impedances reflect valve plane movements. A hemodynamic parameter determining circuit determines at least one hemodynamic parameter based on the impedances reflecting the mechanical functioning of the heart.

### BACKGROUND OF THE INVENTION

1. Field of the Invention

The present invention generally relates to the field of implantable heart stimulation devices, such as pacemakers, implantable cardioverter-defibrillators (ICD), and similar cardiac stimulation devices that also are capable of monitoring and detecting electrical activities and events within the heart. More specifically, the present invention relates to an implantable medical device for monitoring the movements of the valve planes of the heart to determine at least one hemodynamic measure reflecting a mechanical functioning of a heart of a patient.

2. Description of the Prior Art

Implantable heart stimulators that provide stimulation pulses to selected locations in the heart, e.g. selected chambers, have been developed for the treatment of cardiac diseases and dysfunctions. Heart stimulators have also been developed that affect the manner and degree to which the heart chambers contract during a cardiac cycle in order to promote the efficient pumping of blood.

Furthermore, the heart will pump more effectively when a coordinated contraction of both atria and ventricles can be provided. In a healthy heart, the coordinated contraction is provided through conduction pathways in both the atria and the ventricles that enable a very rapid conduction of electrical signals to contractile tissue throughout the myocardium to effectuate the atrial and ventricular contractions. If these conduction pathways do not function properly, a slight or severe delay in the propagation of electrical pulses may arise, causing asynchronous contraction of the ventricles which would greatly diminish the pumping efficiency of the heart. Patients who exhibit pathology of these conduction pathways, such as patients with bundle branch blocks, etc., can thus suffer from compromised pumping performance. For example, asynchronous movements of the valve planes of the right and left side of the heart, e.g. an asynchronous opening and/or closure of the aortic and pulmonary valves, is such an asynchrony that affects the pumping performance in a negative way. This may be caused by right bundle branch block (RBBB), left bundle branch block (LBBB), or A-V block. In a well functioning heart the left and right side of the heart contract more or less simultaneously starting with the contraction of the atria flushing down the blood through the valves separating the atria from the ventricles. In the right side of the heart through the tricuspid valve and in the left side of the heart through the mitral valve. Shortly after the atrial contraction the ventricles contract resulting in increasing blood pressure inside the ventricles that first closes the one way valves to the atria and after that forces the outflow valves to open. In the right side of the heart it is the pulmonary valves, that separates the right ventricle from the pulmonary artery that leads the blood to the lung, which is opened. In the left side of the heart the aortic valve separates the left ventricle from the aorta that transports blood to the whole body. The outflow valves, the pulmonary valve and aortic valve, open when the pressure inside the ventricle exceeds the pressure in the pulmonary artery and aorta, respectively. The ventricles are separated by the intraventricular elastic septum. Hence, for a well functioning heart a substantially synchronous operation of the left and right hand side of the heart, e.g. a synchronous opening and/or closure of the aortic and pulmonary, is of a high importance.

Various procedures have been developed for addressing disorders related to asynchronous function of the heart. For instance, cardiac resynchronization therapy (CRT) can be used for effectuating synchronous atrial and/or ventricular contractions. Furthermore, cardiac stimulators may be provided that deliver stimulation pulses at several locations in the heart simultaneously, such as biventricular stimulators. The stimulation pulses could also be delivered to different locations with a selected delay in an attempt to optimize the hemodynamic performance, e.g. synchronize the closure of the aortic and pulmonary valves, in relation to the specific cardiac dysfunction present at the time of implant.

Information about the mechanical functioning of a heart can be obtained by means electrical signals produced by the heart. In a healthy heart the sinus node, situated in the right atrium, generates electrical signals which propagates throughout the heart and control its mechanical movement. Some medical conditions, however, affect the relationship between the electrical and mechanical activity of the heart and, therefore, measurements of the electrical activity only cannot be relied upon as indicative of the true status of the heart or as suitable for triggering stimulation of the heart.

Consequently, there is a need within the field of methods and devices for obtaining accurate and reliable signals reflecting different aspects of mechanical functioning of the heart.

Impedance measurements has been shown to provide reliable information regarding the mechanical functioning of the heart. Through the impedance measurements, blood volume changes are detectable. Blood has a higher conductivity (lower impedance) than myocardial tissue and lungs. The impedance-volume relationship is inverse; the more blood—the smaller impedance. In EP 1 561 489, for example, transvalvular impedance measurements are made between an atrium and a ventricle electrode of a implanted electro-catheter to provide information indicative of the mechanical state of the heart. The information is used to control the pacing rate of a rate responsive pacemaker. In particular, the impedance between across the tricuspid valve between the atrium and the ventricle of the right hand side of the heart is measured.

However, in order to be able to optimize the functioning of the heart it is of interest to obtain information that provide a more complete picture of the mechanical functioning and the pumping action of the heart and that provide accurate and reliable information of the mechanical functioning and the pumping action of the heart.

### SUMMARY OF THE INVENTION

An object of the present invention is to address the problem of obtaining information that reflects the mechanical functioning and the pumping action of the heart.

A further object of the present invention is to provide a device and method that automatically obtains information that reflects the mechanical functioning and the pumping action of the heart in an accurate and reliable way.

According to an aspect of the present invention, there is provided an implantable medical device for determining at least one hemodynamic measure reflecting a mechanical functioning of a heart of a patient including a pace pulse generator adapted to produce cardiac stimulating pacing pulses and being connectable to at least one medical lead for delivering the pulses to cardiac tissue of the heart. The implantable medical device has an impedance measuring circuit that, during impedance measuring sessions, measures impedance between at least a first pair of electrodes of the at least one medical lead. The at least first pair includes at least one electrode located in an atrium of the heart and at least one valve plane electrode located substantially at the level of a valve plane the heart. The impedance measuring circuit also measures impedance between at least a second pair of electrodes of the at least one medical lead, the at least second pair including at least one electrode located in a ventricle of the heart and at least one valve plane electrode located substantially at the level of the valve plane. These measured impedances reflect valve plane movements. A hemodynamic parameter determining circuit determines at least one hemodynamic parameter based on the impedances, wherein the at least one hemodynamic parameter representing the mechanical functioning of a heart.

According to a second aspect of the present invention, there is provided a method for determining at least one hemodynamic measure reflecting a mechanical functioning of a heart of a patient using an implantable medical device including a pace pulse generator adapted to produce cardiac stimulating pacing pulses and being connectable to at least one medical lead for delivering the pulses to cardiac tissue of the heart. The method includes the steps of, during impedance measuring sessions, measuring impedance between at least a first pair of electrodes of the at least one medical lead, the at least first pair including at least one electrode located in an atrium of the heart and at least one valve plane electrode located substantially at the level of a valve plane the heart, and between at least a second pair of electrodes of the at least one medical lead, the at least second pair including at least one electrode located in a ventricle of the heart and at least one valve plane electrode located substantially at the level of the valve plane. These impedances reflect valve plane movements are obtained. At least one hemodynamic parameter based on the impedances is automatically determined, that reflects the mechanical functioning of a heart.

According to a third aspect of the present invention, there is provided a computer readable medium comprising instructions that cause a programmable device to perform steps of a method according to the second aspect of the present invention.

Thus, the present invention is based on the insight of monitoring valve movements using electrodes placed adjacent to or substantially at the level of the valve plane of the heart by measuring impedance variations between at least one electrode placed adjacent to or substantially at the level of the valve plane and at least one electrode attached in an atrium and at least one electrode attached in a ventricle, respectively. The valve plane movements are caused by the pumping action of the heart, i.e. by the increased and decreased volume of the ventricles, and by studying the valve plane movements the contraction pattern and mechanical functioning of the heart can be monitored. The measured impedances can, in turn, be used to determine hemodynamic parameters reflecting the mechanical functioning of the heart. Thereby, it is possible to automatically obtain information that accurately and reliably reflects the mechanical functioning and the pumping action of the heart.

The obtained information regarding the mechanical functioning of the heart may, for example, be used to optimize parameters of the implantable medical device such as the AV or VV delay. In one embodiment of the present invention, the implantable medical comprises an AV and/or VV delay determining circuit adapted to initiate an optimization procedure, wherein the pace pulse generator is controlled to, based on the hemodynamic parameter, iteratively adjust a present AV and/or VV delay to optimize an AV and/or VV delay with respect to the hemodynamic parameter. Thereby, the AV and/or VV delay can be dynamically and automatically adjusted with respect to the present pumping action of the heart. Further, the adjustments of the AV and/or VV delay can be made dynamically as a response to a changing mechanical functioning of the heart.

In a further embodiment of the present invention, the impedance measuring circuit, during impedance measuring sessions, measures impedance between the at least first pair of electrodes of the at least one medical lead including an electrode located in an atrium of the heart and at least one first valve plane electrode located substantially at the level of the valve plane in close proximity to the right atrium of the heart and at least one second valve plane electrode located substantially at the level of the valve plane in close proximity to the left atrium of the heart, respectively, as well as between the at least second pair of electrodes of the at least one medical lead including an electrode located in a ventricle of the heart and the valve plane electrodes located substantially at the level of the valve plane, respectively. These impedance signals reflect valve plane movements at respective sides of the heart and the hemodynamic parameter determining circuit determines a synchronicity measure based on the impedances, the synchronicity measure reflecting a synchronicity between the valve plane movements of the right hand side and the left hand side of the heart, respectively, during the measurement sessions. In embodiments of the present invention, synchronicity between a closure of the aortic valve and the pulmonary valve and/or an opening of the aortic valve and the pulmonary valve is/are determined.

Thereby, it is possible to monitor the parallelity or synchronicity of the left and right hand side of the heart in an accurate and reliable way as well as the operation of the aortic valve and the pulmonary valves. In a well functioning heart the left and right side of the heart contract more or less simultaneously starting with the contraction of the atria flushing down the blood through the valves separating the atria from the ventricles. In the right side of the heart through the tricuspid valve and in the left side of the heart through the mitral valve. Shortly after the atrial contraction the ventricles contract resulting in increasing blood pressure inside the ventricles that first closes the one way valves to the atria and after that forces the outflow valves to open. In the right side of the heart it is the pulmonary valves, that separates the right ventricle from the pulmonary artery that leads the blood to the lung, which is opened. In the left side of the heart the aortic valve separates the left ventricle from the aorta that transports blood to the whole body. The outflow valves, the pulmonary valve and aortic valve, open when the pressure inside the ventricle exceeds the pressure in the pulmonary artery and aorta, respectively. Hence, for a well functioning heart a substantially synchronous opening and/or closure of the aortic and pulmonary is of a high importance.

In another embodiment of the present invention, a synchronicity between a closure of the mitral valve and the tricuspid valve, respectively, is determined based on the impedances.

According to a further example of the present invention, the AV and/or VV delay determining circuit initiates an optimization procedure, wherein the pace pulse generator is controlled, based on the synchronicity measure, to iteratively adjust a present AV and/or VV delay to identify an AV and/or VV delay that causes substantially synchronized valve plane movements of the right hand side and the left hand side of the heart, respectively, during a cardiac cycle.

In yet another example of the present invention, the AV and/or VV delay determining circuit initiates an optimization procedure, wherein the pace pulse generator is controlled, based on the synchronicity between a closure of the aortic valve and the pulmonary valve and/or the synchronicity between an opening of the aortic valve and the pulmonary valve, to iteratively adjust a present AV and/or VV delay to identify an AV and/or VV delay that causes a substantially synchronized closure and/or opening of the aortic valve and the pulmonary valves.

Moreover, the AV and/or VV delay determining circuit may be adapted to initiate an optimization procedure, wherein the pace pulse generator is controlled to, based on the synchronicity between a closure of the mitral valve and the tricuspid valve, iteratively adjust a present AV and/or VV delay to identify an AV and/or VV delay that causes a substantially synchronized closure of the mitral and tricuspid valves.

In embodiments of the present invention, the impedance measuring circuit determines a maximum and/or minimum impedance of each respective impedance for each cardiac cycle. Moreover, the impedance measuring circuit may be adapted to determine a maximum absolute derivative of each respective impedance for each cardiac cycle.

In still another example, the impedance measuring circuit performs the impedance measuring sessions during successive cardiac cycles, wherein impedance signals reflecting valve plane movements during the successive cardiac cycles are obtained.

According to embodiments of the present invention, the at least one valve plane electrode is placed endocardially.

Alternatively, the at least one valve plane electrode is placed epicardially.

In certain embodiments of the present invention, the at least one valve plane electrode is placed intrapericardially on the surface of the heart.

According to further examples of the present invention, the first valve plane electrode is placed endocardially in the right atrium, or in the left atrium, or in the left ventricle, or in the right ventricle, or epicardially and the second valve plane electrode is placed endocardially in the right atrium, or in the left atrium, or in the left ventricle, or in the right ventricle or epicardially.

As the skilled person realizes, steps of the methods according to the present invention, as well as preferred embodiments thereof, are suitable to realize as computer program or as a computer readable medium.

Further objects and advantages of the present invention will be discussed below by means of exemplifying embodiments.

### BRIEF DESCRIPTION OF THE DRAWINGS

FIG. 1 is a simplified partly cutaway view illustrating an implantable stimulator including an electrode configuration according to the present invention.

FIG. 2 is a simplified partly cutaway view illustrating an electrode configuration according to an embodiment of the present invention.

FIG. 3 is a simplified partly cutaway view illustrating an electrode configuration according to a further embodiment of the present invention.

FIG. 4 is a simplified partly cutaway view illustrating an electrode configuration according to yet another embodiment of the present invention.

FIG. 5 is a simplified partly cutaway view illustrating an electrode configuration according to another embodiment of the present invention.

FIG. 6 is a simplified partly cutaway view illustrating an electrode configuration according to a still another embodiment of the present invention.

FIG. 7 is a simplified partly cutaway view illustrating an electrode configuration according to a further embodiment of the present invention.

FIG. 8 is a simplified partly cutaway view illustrating an electrode configuration according to another embodiment of the present invention.

FIG. 9 is a simplified partly cutaway view illustrating an electrode configuration according to a further embodiment of the present invention.

FIG. 10 is a simplified partly cutaway view illustrating an electrode configuration according to yet another embodiment of the present invention.

FIG. 11 is an illustration in a block diagram form of an implantable stimulator according to the embodiment shown in FIG. 1.

FIG. 12 is a flow chart describing the principles of the present invention according to an embodiment will be described.

### DESCRIPTION OF THE PREFERRED EMBODIMENTS

The following is a description of exemplifying embodiments in accordance with the present invention. This description is not to be taken in limiting sense, but is made merely for the purposes of describing the general principles of the invention. Thus, even though particular types of implantable medical devices such as heart stimulators will be described, e.g. biventricular pacemakers, the invention is also applicable to other types of cardiac stimulators such as dual chamber stimulators, implantable cardioverter defibrillators (ICDs), etc.

In the following a number of different electrode configurations suitable for obtaining impedances reflecting the mechanical functioning of the heart, and in particular movements of the valve plane, will be discussed.

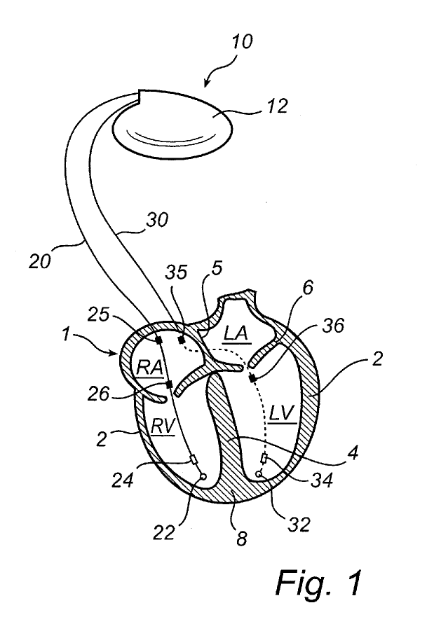

With reference first to FIG. 1, there is shown a implantable medical device according to an embodiment of the present invention. According to this embodiment, the invention is implemented in a stimulation device 10. The stimulation device 10 is in electrical communication with a patient's heart 1 by two leads 20 and 30 suitable for delivering multi-chamber stimulation, which leads 20 and 30 are connectable to the stimulator 10. The illustrated portions of the heart 1 include right atrium RA, the right ventricle RV, the left atrium LA, the left ventricle LV, cardiac walls 2, the ventricle septum 4, the valve plane 6, and the apex 8. The valve plane 6 refers to the annulus fibrosis plane separating the ventricle from the atria and containing all four heart valves, i.e. the aortic, pulmonary, mitral, and tricuspid valves.

In order to sense right ventricular and atrium cardiac signals and impedances and to provide stimulation therapy to the right ventricle RV, the stimulation device 10 is coupled to an implantable right ventricular lead 20 having a ventricular tip electrode 22, a ventricular annular or ring electrode 24, and a first valve plane electrode 26. The ring electrode 24 is arranged for sensing electrical activity, intrinsic or evoked, in the right ventricle RV. The right ventricular tip electrode 22 is arranged to be implanted in the endocardium of the right ventricle, e.g. near the apex 8 of the heart. Thereby, the tip electrode 22 becomes attached to cardiac wall. In this example, the tip electrode 22 is fixedly mounted in a distal header portion of the lead 20. Furthermore, the first valve plane electrode 26, which may a annular or ring electrode, is located substantially at the level of the valve plane 6.

In order to sense left atrium and ventricular cardiac signals and impedances and to provide pacing therapy for the left ventricle LV, the stimulation device 10 is coupled to a “coronary sinus” lead 30 designed for placement via the coronary sinus in veins located distally thereof, so as to place a distal electrode adjacent to the left ventricle and an electrode adjacent to the right atrium RA. The coronary sinus lead 30 is designed to received ventricular cardiac signals from the cardiac stimulator 10 and to deliver left ventricular LV pacing therapy using at least a left ventricular tip electrode 32 to the heart 1. In the illustrated example, the LV lead 30 has an annular ring electrode 34 for sensing electrical activity related to the left ventricle LV of the heart. Moreover, a second valve plane electrode 36, which may a annular or ring electrode, is located substantially at the level of the valve plane 6 and measurement electrode 35, which may a annular or ring electrode, is located adjacent to the right atrium RA.

With reference to FIG. 1, the impedances that can be detected by means of the illustrated embodiment will be described. At the right side of the heart 1, the impedance Z₂₆₋₂₂ between right ventricular tip electrode 22 and the valve plane electrode 26 and the impedance Z₂₆₋₃₅ between the valve plane electrode 26 and the electrode 35 located adjacent to the right atrium RA can be detected, respectively. Furthermore, at the left hand side of the heart 1, the impedance Z₃₆₋₃₂ between the left ventricular tip electrode 32 and the valve plane electrode 36 and the impedance Z₃₆₋₃₅ between the valve plane electrode 36 and the electrode 35 located adjacent to the right atrium RA can be detected, respectively. Since the electrode 35 located adjacent to the right atrium RA and the ventricle electrodes 22 and 32 essentially do not move during the cardiac cycle, the variation in the impedances are mainly due to movements of the valve plane 6.

Thus, the impedance Z₂₆₋₃₅ and the impedance Z₂₆₋₂₂, respectively, will vary during the cardiac cycle as a response to the movements of the valve plane 6 at the right hand side of the heart 1. Similarly, at the left hand side of the heart 1, the impedance Z₃₆₋₃₅ the impedance Z₃₆₋₃₂ will vary during the cardiac cycle as a response to the movements of the valve plane 6.

Moreover, the impedance Z₃₆₋₂₂ will be substantially constant over the cardiac cycle, which also is the case for the impedance Z₃₆₋₃₂. By comparing the detected impedances of the respective sides of the heart, asynchronicity or parallelity of the valve plane movements of the respective sides of the valve plane 6 can be determined. In case of an asynchronous depolarization sequence of the heart, the valve plane may move asynchronously and be bent during the heart cycle which will be reflected by an asynchronicity between the detected impedance at the right hand side and the left hand side, respectively.

Turning briefly to FIGS. 2-9, alternative embodiments for placement of cardiac leads, and cardiac electrodes are illustrated. In FIG. 2, an embodiment including two endocardially positioned leads 41 and 42 connected to a stimulation device (see FIG. 1) comprising an atrial distal tip electrode 44 located in the right atrium RA and a ventricular distal tip electrode 43 located in the right ventricle RV, respectively. The leads 41 and 42 can be fixedly attached to the cardiac wall according to conventional practice. Thereby, the electrodes 43 and 44 will be essentially immobile during the cardiac cycle. A third electrode 45 is located epicardially and is connected to the stimulation device by means of a lead 46. The lead 46 and the electrode 45 can be located epicardially by means of, for example, intrapericardial implantation technique. The electrode 45 is placed at the level of the valve plane 6. Thereby, the variation in the impedance Z₄₅₋₄₃ between the electrode 45 placed at the level of the valve plane 6 and the electrode 43 placed in the right ventricle RV and the impedance Z₄₅₋₄₄ between the electrode 45 placed at the level of the valve plane 6 and the electrode 44 placed in the right atrium RA, respectively, are mainly due to movements of the valve plane. The impedance Z₄₄₋₄₃ between the electrode 44 placed in the right atrium RA and the electrode 43 placed in the right ventricle RV will essentially by the same over a cardiac cycle since the electrodes 43, 44 are essentially immobile during the cardiac cycle.

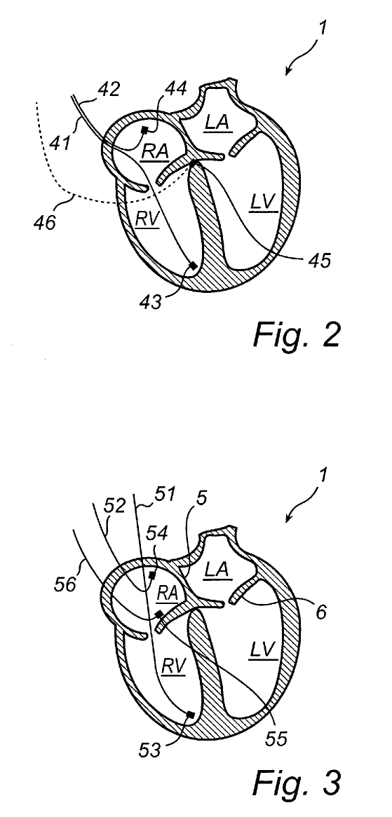

With reference now to FIG. 3, an embodiment in which the impedances are measured by means of three endocardially placed electrodes. Similar to the embodiment shown in FIG. 2, two endocardially positioned leads 51 and 52 connected to a stimulation device (see FIG. 1) having an atrial distal tip electrode 54 located in the right atrium RA and a ventricular distal tip electrode 53 located in the right ventricle RV, respectively. The leads 51 and 52 can be fixedly attached to the cardiac wall according to conventional practice. Thereby, the electrodes 53 and 54 will be essentially immobile during the cardiac cycle. The third electrode 55 is also located endocardially in the right atrium RA and is connected to the stimulation device by means of a lead 46, transvenously advanced through a vein to the inside of the heart 1. The electrode 55 is placed at the level of the valve plane 6. Thereby, the variation in the impedance Z₅₅₋₅₃ between the electrode 55 placed at the level of the valve plane 6 in the right atrium RA and the electrode 53 placed in the right ventricle RV and the impedance Z₅₅₋₅₄ between the electrode 55 placed at the level of the valve plane 6 in the right atrium RA and the electrode 54 placed in the upper part of the right atrium RA, respectively, is mainly due to movements of the valve plane 6. The impedance Z₅₄₋₅₃ between the electrode 54 placed in the upper part of the right atrium RA and the electrode 53 placed in the right ventricle RV will essentially be the same over a cardiac cycle since the electrodes 53, 54 are essentially immobile during the cardiac cycle.

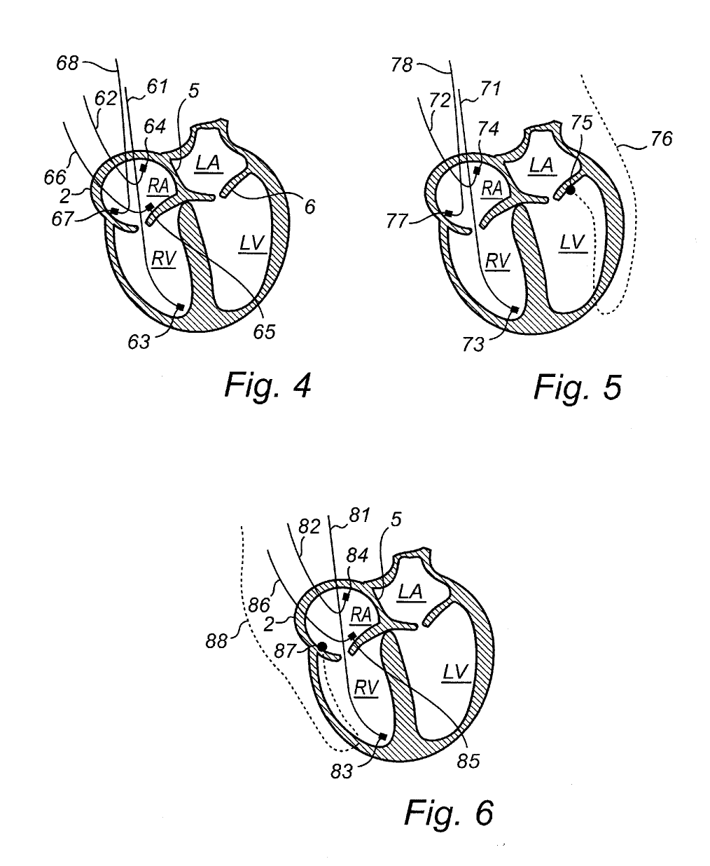

Referring now to FIG. 4, yet another electrode configuration for measuring impedances reflecting the valve plane movements will be described. According to this embodiment, two endocardially positioned leads 61 and 62 connected to a stimulation device (see FIG. 1) comprising an atrial distal tip electrode 64 located in the right atrium RA and a ventricular distal tip electrode 63 located in the right ventricle RV, respectively. The leads 61 and 62 can be fixedly attached to the cardiac wall according to conventional practice. Thereby, the electrodes 63 and 64 will be essentially immobile during the cardiac cycle. Further, two electrodes are placed at the level of the valve plane 6 endocardially in this configuration. A first valve plane electrode 65 is placed endocardially at the level of the valve plane 6 in the right atrium RA adjacent to atrial septum 5 and a second valve plane electrode 67 is placed endocardially at the level of the valve plane in the right atrium RA adjacent to the cardiac wall 2. The first and second valve plane electrodes 65, 67 are arranged at leads 66, 68 transvenously advanced through a vein to the inside of the heart 1 and connected to the stimulation device (see FIG. 1). The variations in the impedance Z₆₅₋₆₃ between the electrode 65 placed at the level of the valve plane 6 in the right atrium RA adjacent to the atrial septum 5 and the electrode 63 placed in the right ventricle RV and the impedance Z₆₅₋₆₄ between the electrode 65 placed at the level of the valve plane 6 in the right atrium RA adjacent to the atrial septum 5 and the electrode 64 placed in the upper part of the right atrium RA, respectively, are mainly due to movements of the valve plane 6. Similarly, the variations in the impedance Z₆₇₋₆₃ between the electrode 67 placed at the level of the valve plane 6 in the right atrium RA adjacent to the cardiac wall 2 and the electrode 63 placed in the right ventricle RV and the impedance Z₆₇₋₆₄ between the electrode 67 placed at the level of the valve plane 6 in the right atrium RA adjacent to the cardiac wall 2 and the electrode 64 placed in the upper part of the right atrium RA, respectively, are mainly due to movements of the valve plane 6. The impedance Z₆₄₋₆₃ between the electrode 64 placed in the upper part of the right atrium RA and the electrode 63 placed in the right ventricle RV will essentially by the same over a cardiac cycle since the electrodes 63, 64 are essentially immobile during the cardiac cycle.

In FIG. 5, a further embodiment is shown. Two leads 71 and 72 are endocardially positioned and connected to a stimulation device (see FIG. 1) comprising an atrial distal tip electrode 74 located in the right atrium RA and a ventricular distal tip electrode 73 located in the right ventricle RV, respectively. The leads 71 and 72 can be fixedly attached to the cardiac wall according to conventional practice. Thereby, the electrodes 73 and 74 will be essentially immobile during the cardiac cycle. Two electrodes are placed at the level of the valve plane 6 in this configuration. A first valve plane electrode 75 is placed epicardially at the level of the valve plane 6 at the left hand side of the heart 1 and a second valve plane electrode 77 is placed endocardially at the level of the valve plane in the right atrium RA adjacent to the cardiac wall 2. The first valve plane 75 is arranged on a lead 76 located epicardially by means of, for example, intrapercardial implantation technique, and connected to the stimulation device (see FIG. 1). The second valve plane electrode 77 is arranged at a lead 78 transvenously advanced through a vein to the inside of the heart 1 and connected to the stimulation device (see FIG. 1). The variations in the impedance Z₇₅₋₇₃ between the electrode 65 placed at the level of the valve plane 6 and the electrode 73 placed in the right ventricle RV and in the impedance Z₇₅₋₇₄ between the electrode 75 placed at the level of the valve plane 6 on the left hand side of the heart 1 and the electrode 74 placed in the upper part of the right atrium RA, respectively, are mainly due to movements of the valve plane 6 on the left hand side of the heart 1. Similarly, the variations in the impedance Z₇₇₋₇₃ between the electrode 77 placed at the level of the valve plane 6 in the right atrium RA adjacent to the cardiac wall 2 and the electrode 73 placed in the right ventricle RV and the impedance Z₇₇₋₇₄ between the electrode 77 placed at the level of the valve plane 6 in the right atrium RA adjacent to the cardiac wall 2 and the electrode 74 placed in the upper part of the right atrium RA, respectively, are mainly due to movements of the valve plane 6 at the right hand side of the heart 1. By comparing the impedance Z₇₇₋₇₄ and the Z₇₇₋₇₃ it is possible to monitor and detect the parallelity or synchronism between the valve plane movements at the respective sides of the heart. The impedance Z₇₄₋₇₃ between the electrode 74 placed in the upper part of the right atrium RA and the electrode 73 placed in the right ventricle RV will essentially by the same over a cardiac cycle since the electrodes 73, 74 are essentially immobile during the cardiac cycle.

Turning to FIG. 6, a further embodiment of the present invention will be described. Two leads 81 and 82 are endocardially positioned, as in the embodiment shown in FIG. 5, comprising an atrial distal tip electrode 84 located in the right atrium RA and a ventricular distal tip electrode 83 located in the right ventricle RV, respectively. The leads 81 and 82 can be fixedly attached to the cardiac wall according to conventional practice. Thereby, the electrodes 83 and 84 will be essentially immobile during the cardiac cycle. Further, a first valve plane electrode 85 is placed endocardially at the level of the valve plane in the right atrium RA adjacent to the atrial septum 5 and a second valve plane electrode 87 is placed epicardially at the level of the valve plane 6 adjacent to the cardiac wall 2. The first valve plane electrode 85 is arranged at a lead 86 transvenously advanced through a vein to the inside of the heart 1 and connected to the stimulation device (see FIG. 1). The second valve plane 87 is arranged on a lead 88 located epicardially by means of, for example, intrapercardial implantation technique, and connected to the stimulation device (see FIG. 1). The electrode configurations of this embodiment is similar to the electrode configurations shown in FIG. 4 and thus the measured impedances will be similar to the impedances measured using the configuration shown in FIG. 4 for what reason a detailed description thereof is omitted.

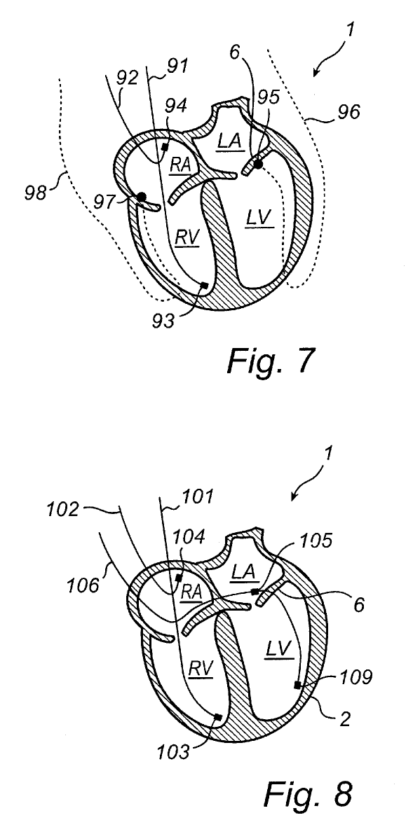

In FIG. 7, another configuration of electrodes for measuring the valve plane movements by means of impedances will be shown. As can be seen, two leads 91 and 92 are endocardially positioned, as in the embodiment shown in FIG. 5, having an atrial distal tip electrode 94 located in the right atrium RA and a ventricular distal tip electrode 93 located in the right ventricle RV, respectively. The leads 91 and 92 can be fixedly attached to the cardiac wall according to conventional practice. Thereby, the electrodes 93 and 94 will be essentially immobile during the cardiac cycle. Further, a first valve plane electrode 95 is placed epicardially at the level of the valve plane 6 at the left hand side of the heart 1 and a second valve plane electrode 97 is placed epicardially at the level of the valve plane 6 at the right hand side of the heart 1. The first valve plane electrode 95 and the second valve plane 97, respectively, are arranged on a leads 96, 98 located epicardially by means of, for example, intrapercardial implantation technique, and are connected to the stimulation device (see FIG. 1), respectively. The electrode configurations of this embodiment is similar to the electrode configurations shown in FIG. 5 and thus the measured impedances will be similar to the impedances measured using the configuration shown in FIG. 5 for what reason the description thereof is omitted.

Referring now to FIG. 8, yet another embodiment of the present invention will be discussed. Two leads 101 and 92 are endocardially positioned comprising an atrial distal tip electrode 104 located in the right atrium RA and a ventricular distal tip electrode 103 located in the right ventricle RV, respectively. The leads 101 and 102 can be fixedly attached to the cardiac wall according to conventional practice. Thereby, the electrodes 103 and 104 will be essentially immobile during the cardiac cycle. In addition, a left ventricle lead 106 is placed in a left lateral coronary vein, advanced from the right atrium RA through the coronary sinus. The left ventricle lead 106 comprises a left ventricle tip electrode 109 and a valve plane electrode 105, e.g. an annular or ring electrode, located adjacent to the valve plane 6. The left ventricle lead 106 may be fixated at the cardiac wall using conventional practice. In this configuration, the valve plane movements detected will be substantially the valve plane movements at the left hand side of the heart 1. The impedance Z₁₀₅₋₁₀₃ between the electrode 105 placed at the level of the valve plane 6 and the electrode 103 placed in the right ventricle RV and in the impedance Z₁₀₅₋₁₀₄ between the electrode 105 placed at the level of the valve plane 6 on the left hand side of the heart 1 and the electrode 104 placed in the upper part of the right atrium RA, respectively, are measured and the variations are mainly due to movements of the valve plane 6 on the left hand side of the heart 1. Alternatively, the impedances Z₁₀₅₋₁₀₉ between the electrode 105 placed at the level of the valve plane 6 and the left ventricle electrode 109 and the impedance Z₁₀₅₋₁₀₃ between the electrode 105 placed at the level of the valve plane 6 and the electrode 103 placed in the right ventricle RV, respectively, can be measured.

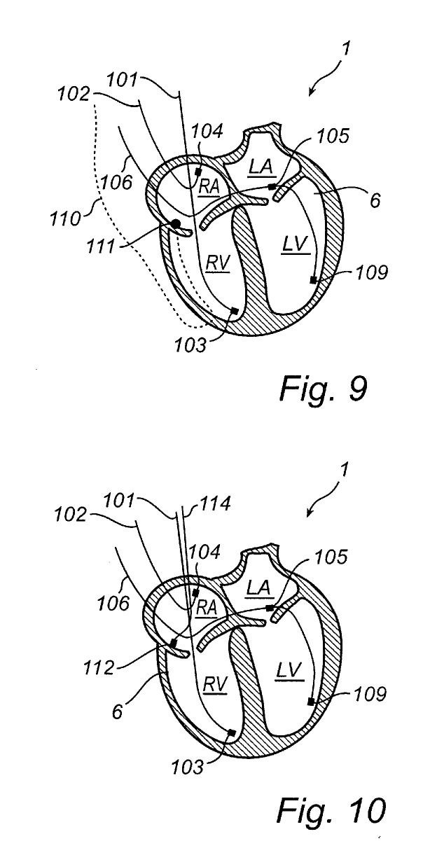

In FIGS. 9 and 10, alternative electrode configurations to the configuration illustrated in FIG. 8 are shown. As can be seen in FIG. 8, a further lead 110 including a valve plane electrode 111 is located epicardially at the right hand side of the heart 1 by means of, for example, intrapercardial implantation technique, and connected to the stimulation device (see FIG. 1). Thereby, the impedances can be measured in a similar way as in the embodiment described with reference to FIG. 5 and it is possible to monitor and detect the degree of parallel operation or synchronism between the valve plane movements at the respective sides of the heart 1. In the embodiment shown in FIG. 9, a further lead 114 is transvenously advanced through a vein to the inside of the heart 1 and connected to the stimulation device (see FIG. 1), which lead 114 includes a second valve plane electrode 112 placed endocardially at the level of the valve plane in the right atrium RA adjacent to the cardiac wall 2. Thereby, the impedances can be measured in a similar way as in the embodiment described with reference to FIG. 5 and it is possible to monitor and detect the parallelity or synchronism between the valve plane movements at the respective sides of the heart 1. Thereby, the impedances can be measured in a similar way as in the embodiment described with reference to FIG. 5 and it is possible to monitor and detect the parallelity or synchronism between the valve plane movements at the respective sides of the heart 1.

Even though a number of examples have been illustrated in FIGS. 1-10, the invention is not restricted the illustrated lead and electrode configurations and placements. For example, more epicardial electrodes can be placed at the level of the valve plane. Furthermore, different types of sensors can also be arranged in the leads to obtain other types of information. For example, cardiac wall motion sensors of accelerometer type can be arranged in a right ventricle and/or a left ventricle lead.

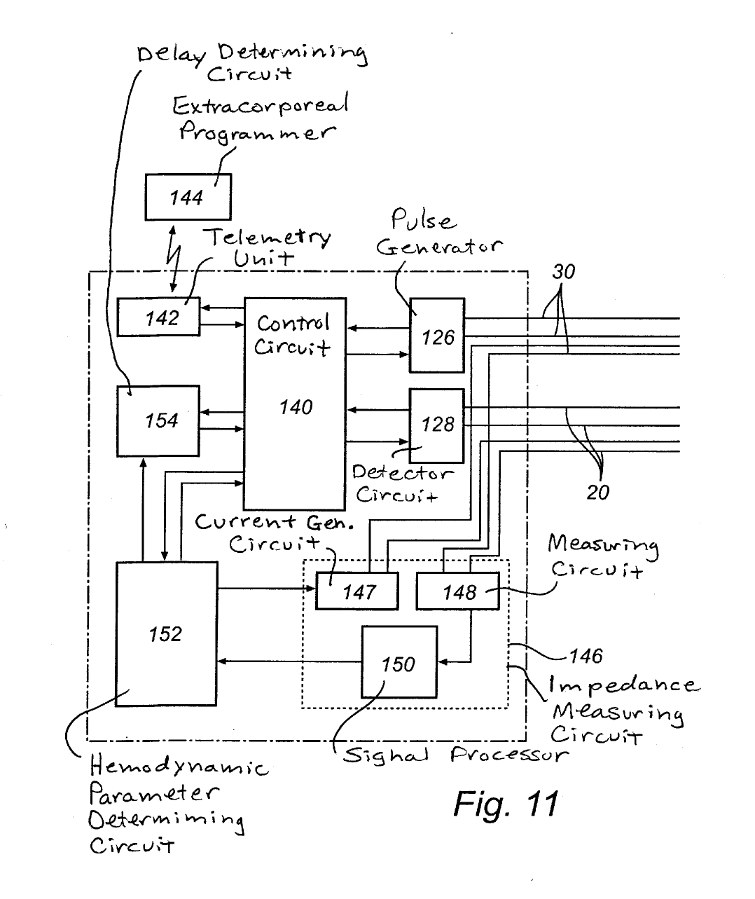

Turning now to FIG. 11, the heart stimulator 10 of FIG. 1 is shown in a block diagram form. For illustrative purposes, reference is made to FIG. 1 for the elements of the leads that are intended for positioning in or at the heart. The heart stimulator 10 is connected to a heart 1 order to sense heart signals and emit stimulation pulses to the heart 1. Electrodes located within and at the heart, for example, any one of the electrode configurations illustrated in FIGS. 1-10 and outside the heart, for example, an indifferent electrode 12 (which, in this instance, is formed by the enclosure of the heart stimulator 10 but can also is formed by a separate electrode located somewhere in the body) are connected to a pulse generator 126 in the heart stimulator 10. The electrodes located within and/or at the heart are connected to the stimulator 10 via leads, for example, the leads 20 and 30 shown in FIG. 1. A detector 128 is also connected to the electrodes in order to sense activity of the heart.

The pulse generator 126 and the detector 128 are controlled by a control unit 140 which regulates the stimulation pulses with respect to amplitude, duration and stimulation interval, the sensitivity of the detector 128 etc.

A physician using an extracorporeal programmer 144 can communicate, via a telemetry unit 142, with the heart stimulator 10 and thereby obtain information on identified conditions and also reprogram the different functions of the heart stimulator 10.

Furthermore, the heart stimulator 10 has an impedance measuring circuit 146 adapted to, during impedance measuring sessions, measure impedance signals between at least a first pair of electrodes, which at least first pair includes at least one electrode located in an atrium of the heart and at least one valve plane electrode located substantially at the level of a valve plane the heart. Further, the impedance measuring circuit 146 is adapted to, during the impedance measuring sessions, measure impedance signals between at least a second pair of electrodes, which at least second pair includes at least one electrode located in at least one ventricle of the heart and at least one valve plane electrode located substantially at the level of the valve plane. In FIGS. 1-10, a number of different electrode configurations by which impedance signals reflecting the valve plane movements can be obtained are shown. The impedance measuring circuit 146 includes a current generating circuit 147 adapted to apply excitation current pulses between the respective electrode pairs and a measuring circuit 148 adapted to measure the resulting voltage over the respective electrode pairs and determined resulting impedance signals. An impedance signal processor 150 is connected to the measuring circuit 148 and is adapted to process the impedance signals to determine respective impedances over each measurement session for the respective electrode pairs. The impedance signal processor 150 may be adapted to determine maximum or minimum impedances over a cardiac cycle, for example, for respective atrium and/or respective ventricle and/or to determine maximum absolute derivative of the impedance for respective atrium and/or respective ventricle.

Moreover, the heart stimulator 10 has a hemodynamic parameter determining circuit 152 adapted to determine at least one hemodynamic parameter based on impedances received from the impedance measuring circuit 146. The hemodynamic parameter determining circuit 152 includes a microprocessor, which may, for example, control the impedance measuring circuit 146 to, inter alia, initiate an impedance measuring session, the length and/or amplitude of the generated current pulses. The at least one hemodynamic parameter based on the measured impedances reflects the mechanical functioning of the heart. A number of different parameters may be extracted from the measured impedances and monitored including pre-ejection period, a contraction patter, mitral regurgitation, a synchronicity between the left and right hand sides of the heart, etc.

In one embodiment, the hemodynamic parameter determining circuit 152 is adapted to determine a synchronicity measure based on the impedances reflecting the synchronicity between the valve plane movements of the right hand side and the left hand side of the heart, respectively, during impedance measurement sessions. Through the impedance measurements, blood volume changes are detected. Blood has a higher conductivity (lower impedance) than myocardial tissue and lungs. The impedance-volume relationship is inverse; the more blood—the smaller impedance. Accordingly, the impedance will vary over the cardiac cycle in connection with the contraction and filling of the atria and ventricles, respectively, in, hence, in connection with the pressure variations during the cycle. For example, the ventricle volume is at a maximum level at the onset of the systolic phase of the ventricles, which corresponds to a minimum impedance measured over the ventricles, and the ventricle volume is at a minimum level at onset of diastolic phase of the ventricles, which corresponds to a maximum impedance measured over the ventricles.

In one embodiment, the synchronicity between a closure of the aortic valve and the pulmonary valve and/or a synchronicity between an opening of the aortic valve and the pulmonary valve is determined using the measured impedances. For example, if the electrode configuration illustrated in FIG. 5 is used, the impedance Z₇₅₋₇₃ between the electrode 75 placed at the level of the valve plane 6 and the electrode 73 placed in the right ventricle RV reflects the movements of the valve plane 6 on the left hand side of the heart 1 and the variations of the blood volume of the right ventricle RV. Similarly, the variations in the impedance Z₇₇₋₇₃ between the electrode 77 placed at the level of the valve plane 6 in the right atrium RA adjacent to the cardiac wall 2 and the electrode 73 placed in the right ventricle RV reflects the movements of the valve plane 6 at the right side of the heart 1 and the variations of the blood volume of the left ventricle LV. By comparing the impedance Z₇₅₋₇₃ and the impedance Z₇₇₋₇₃ it is possible to detect an asynchronism between the valve plane movements at the respective sides of the heart, for example, the opening and/or closure of the aortic and pulmonary valves. For example, peak amplitudes of the impedances, maximum absolute derivative, or morphology of the impedance curves over a cardiac cycle can be studied to detect such an asynchronism.

The heart stimulator 10 further has an AV and/or VV delay determining circuit 154 adapted to determine a AV and/or VV delay with respect to a determined hemodynamic parameter, for example, a synchronicity between an opening and/or a closure of the pulmonary and aortic valves. In one embodiment, the AV and/or VV delay determining circuit 154 is integrated in the control circuit 140. The AV and/or VV delay determining circuit 154 is adapted to initiate an optimization procedure (e.g. via the control circuit 140), wherein the pace pulse generator 126 is controlled to iteratively adjust a present AV and/or VV delay to optimize an AV and/or VV delay with respect to the determined hemodynamic parameter starting from the determined AV and/or VV delay. For example, if it is determined that the movements of the valve plane at the right hand side of the heart is ahead the movements of the left hand side in the cardiac cycle, the VV delay may be adjusted such that the left ventricle is stimulated first and vice versa. However, it is important that AV delay of the left side has a sufficient length, i.e. if the different between the right side and the left side is large there is a risk that the effective AV delay on the left side becomes too short. In such a case, the AV delay should also be lengthened.

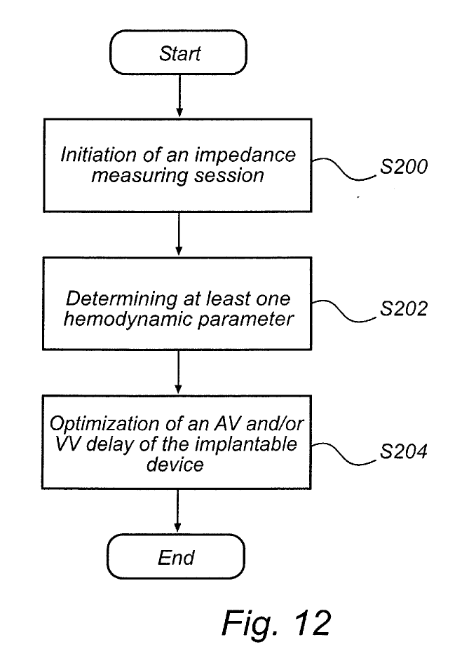

Turning now to FIG. 12, the principles of the present invention according to an embodiment will be described. First, at step 200, a request for an initiation of impedance measuring session is received by the hemodynamic parameter determining circuit 152. This request may be received from the control circuit 140 or from an external device via the telemetry unit 142. Alternatively, the hemodynamic parameter determining circuit 152 may be adapted to automatically initiate the impedance measuring sessions at regular intervals. The hemodynamic parameter determining circuit 152 sends an initiation signal to the impedance measuring circuit 146. This step may be preceded by a check of the measuring conditions, for example, which posture the patient is in, or the activity level. This measuring condition information may be used in the optimization of the AV and/or VV delay. Upon receipt of the initiation signal, which may include information regarding, for example, current pulse width or current amplitude, the impedance measuring circuit 146 measures, during impedance measuring sessions, impedances between at least a first pair of electrodes of the at least one medical lead, the at least first pair including at least one electrode located in an atrium of the heart and at least one valve plane electrode located substantially at the level of a valve plane the heart, and between at least a second pair of electrodes of the at least one medical lead, the at least second pair including at least one electrode located in a ventricle of the heart and at least one valve plane electrode located substantially at the level of the valve plane, wherein impedances reflecting valve plane movements are obtained, and in FIGS. 1-10, a number of conceivable electrode configurations are illustrated. Thereafter, at step 202, at least one hemodynamic parameter based on the impedances, wherein the at least one hemodynamic parameter reflects the mechanical functioning of a heart, is determined. According to embodiments, a synchronicity measure is determined, which reflects a parallelity or synchronicity between the left and right side of the heart. In particular, a parallelity or synchronicity between the valve planes at the left and right side of the heart can be monitored by means of the impedances, e.g. the synchronicity between an opening and/or closure of the aortic and pulmonary valves. Then, at step 204, an optimization procedure is initiated including adjusting present AV and/or VV delay to optimize an AV and/or VV delay with respect to the hemodynamic parameter.

In further embodiments, the indication of an asynchronicity between valve plane movements of the right and left side of the heart, for example, between the opening and/or closing of the aortic and pulmonary valves, could be used for triggering an alarm signal to the patient. This alarm signal could be intended for prompting the patient to seek medical assistance for care of follow-up. The alarm signal may alternatively, or as a compliment, be transferred to an extracorporeal unit 144 or a care institution via the telemetry unit 142.

Although modifications and changes may be suggested by those skilled in the art, it is the intention of the inventors to embody within the patent warranted hereon all changes and modifications as reasonably and properly come within the scope of their contribution to the art.

We claim as our invention:

1-29. (canceled)

30. An implantable medical device comprising:
a pacing pulse generator that generates cardiac stimulation pacing pulses;
a medical lead connected to said pulse generator and adapted for implantation in a subject to deliver the cardiac stimulating pacing pulses to cardiac tissue in vivo, said medical lead comprising at least a first pair of electrodes including at least one electrode adapted for location in an a atrium of the heart and at least one valve plane electrode adapted for location substantially at a level of a valve plane of the heart, and at least a second pair of electrodes comprising at least one electrode adapted for location in a ventricle of the heart and at least one electrode adapted for location at the level of said valve plane;
an impedance measuring circuit connected to said medical lead that senses impedances reflecting movement of said valve plane by measuring an impedance between said first pair of electrodes and an impedance between said second pair of electrodes; and
a hemodynamic parameter determining circuit supplied with said impedances measured by said impedance measuring circuit and configured to determine at least one hemodynamic parameter based on said impedances measured by said impedance measuring circuit, that represents mechanical functioning of the heart, said hemodynamic parameter determining circuit making said hemodynamic parameter available at an output thereof.

31. An implantable medical device as claimed in claim 30 comprising a delay determining circuit that operates said pacing pulse generator to cause successive stimulation pulses to be emitted by said pulse generator with a delay therebetween, selected from the group consisting of an AV delay and a VV delay, said delay determining circuit being supplied with said hemodynamic parameter from said hemodynamic determining circuit and being configured to iteratively adjust said delay to optimize said delay with respect to said hemodynamic parameter.

32. An implantable medical device as claimed in claim 30 wherein:
said medical lead comprises two valve plane electrodes, including a first valve plane electrode adapted for location substantially at the level of said valve plane in close proximity to the right atrium of the heart and at least one second valve plane electrode adapted for location substantially at the level of said valve plane in close proximity to the left atrium of the heart;
said impedance measuring circuit measures impedance between respective first pairs of electrodes comprising a first first pair of electrodes formed by said electrode located in the atrium of the heart and said first valve plane electrode and a second first pair of electrodes comprising said electrode in the atrium and said second valve plane electrode to measure an impedance reflecting valve plane movements at a first side of said valve plane, and wherein said impedance measuring circuit measures impedance between two second pairs of electrodes including a first second pair comprising said electrode in the ventricle and said first valve plane electrode, and a second second pair comprising said electrode in the ventricle and said second valve plane electrode, to measure impedance reflecting valve plane movements at a second side of said valve plane, opposite said first side; and
said hemodynamic parameter determining circuit being configured to determine a synchronicity measure based on said impedances respectively measured at said first and second sides of said valve plane, said synchronicity measure reflecting synchronism between the valve plane movements at said first and second sides of the valve plane, and said hemodynamic determining circuit making said synchronicity measure available at said output thereof.

33. An implantable medical device as claimed in claim 32 wherein said hemodynamic parameter determining circuit is configured to determine said synchronicity measure from the group consisting of synchronicity between closure of the aortic valve and closure of the pulmonary valve, and synchronicity between opening of the aortic valve and opening of the pulmonary valve.

34. An implantable medical device as claimed in claim 32 wherein said hemodynamic parameter determining circuit is configured to determine said synchronicity measure from the group consisting of synchronicity between closure of the mitral valve and closure of the tricuspid valve.

35. An implantable medical device as claimed in claim 34 comprising:
a delay determining circuit that operates said pacing pulse generator to cause successive stimulation pulses to be emitted by said pulse generator with a delay therebetween, selected from the group consisting of an AV delay and a VV delay, said delay determining circuit being supplied with said hemodynamic parameter from said hemodynamic determining circuit and being configured to iteratively adjust said delay to optimize said delay with respect to said hemodynamic parameter; and
said delay determining circuit being supplied with said synchronicity measure from said hemodynamic parameter determining circuit and being configured to iteratively adjust said delay to produce substantially synchronized movements of said valve plane at said first side and said second side during a cardiac cycle.

36. An implantable medical device as claimed in claim 32 wherein said first valve plane electrode is configured for placement at a placement site selected from the group consisting of endocardially in the right atrium, endocardially in the left atrium, endocardially in the left ventricle, endocardially in the right ventricle, and epicardially, and wherein said second valve plane electrode is configured for placement at a placement site selected from the group consisting of endocardially in the right atrium, endocardially in the left atrium, endocardially in the left ventricle, endocardially in the right ventricle, and epicardially.

37. An implantable medical device as claimed in claim 30 wherein said impedance measuring circuit measures, in each cardiac cycle, an extreme value of said impedance between said at least one first pair of electrodes and said impedance between said at least one second pair of electrodes, said extreme value being selected from the group consisting of a maximum and a minimum.

38. An implantable medical device as claimed in claim 30 wherein said impedance measuring circuit determines an absolute value of a maximum derivative with respect to time for each impedance, in each cardiac cycle, between said at least one first pair of electrodes and between said at least one second pair of electrodes.

39. An implantable medical device as claimed in claim 30 wherein said impedance measuring circuit measures said impedance between each of said at least one first pair of electrodes and at least one second pair of electrodes during successive cardiac cycles.

40. An implantable medical device as claimed in claim 30 wherein said at least one valve plane electrode is an electrode configured for endocardial placement.

41. An implantable medical device as claimed in claim 30 wherein said at least one valve plane electrode is an electrode configured for epicardial placement.

42. A method for operating an implantable medical device comprising the steps of:
from a pacing pulse generator, emitting cardiac stimulation pacing pulses;
implanting a medical lead connected to said pulse generator to deliver the cardiac stimulating pacing pulses to cardiac tissue in vivo, via at least a first pair of electrodes carried by said medical lead including at least one electrode placed in an atrium of the heart and at least one valve plane electrode placed substantially at a level of a valve plane of the heart, and via at least a second pair of electrodes including at least one electrode placed in a ventricle of the heart and at least one electrode placed at the level of said valve plane;
with an impedance measuring circuit connected to said medical lead, sensing impedances reflecting movement of said valve plane by measuring an impedance between said first pair of electrodes and an impedance between said second pair of electrodes; and
in a hemodynamic parameter determining circuit supplied with said impedances measured by said impedance measuring circuit, automatically determining at least one hemodynamic parameter based on said impedances measured by said impedance measuring circuit, that represents mechanical functioning of the heart, and making said hemodynamic parameter available at an output of said hemodynamic parameter determining circuit.

43. A method as claimed in claim 42 comprising:
from a delay determining circuit, operating said pacing pulse generator to cause successive stimulation pulses to be emitted by said pulse generator with a delay therebetween, selected from the group consisting of an AV delay and a VV delay;
supplying said delay determining circuit with said hemodynamic parameter from said hemodynamic determining circuit and, in said delay determining circuit, iteratively adjusting said delay to optimize said delay with respect to said hemodynamic parameter.

44. A method as claimed in claim 42 comprising:
providing said medical lead with two valve plane electrodes, including a first valve plane electrode placed substantially at the level of said valve plane in close proximity to the right atrium of the heart and at least one second valve plane electrode placed substantially at the level of said valve plane in close proximity to the left atrium of the heart;
with said impedance measuring circuit, measuring impedance between respective first pairs of electrodes comprising a first first pair of electrodes formed by said electrode in the atrium of the heart and said first valve plane electrode and a second first pair of electrodes carried by said medical lead including said electrode in the atrium and said second valve plane electrode, to measure an impedance reflecting valve plane movements at a first side of said valve plane;
with said impedance measuring circuit, measuring impedance between two second pairs of electrodes including a first second pair including said electrode in the ventricle and said first valve plane electrode, and a second second pair comprising said electrode in the ventricle and said second valve plane electrode, to measure impedance reflecting valve plane movements at a second side of said valve plane, opposite said first side; and
in said hemodynamic parameter determining circuit, determining a synchronicity measure based on said impedances respectively measured at said first and second sides of said valve plane, said synchronicity measure reflecting synchronism between the valve plane movements at said first and second sides of the valve plane, and making said synchronicity measure available at said output of said hemodynamic determining circuit.

45. A method as claimed in claim 44 comprising, in said hemodynamic parameter determining circuit, determining said synchronicity measure from the group consisting of synchronicity between closure of the aortic valve and closure of the pulmonary valve, and synchronicity between opening of the aortic valve and opening of the pulmonary valve.

46. A method as claimed in claim 44 comprising in said hemodynamic parameter determining circuit, determining said synchronicity measure from the group consisting of synchronicity between closure of the mitral valve and closure of the tricuspid valve.

47. A method as claimed in claim 46 comprising:
from a delay determining circuit, that operating said pacing pulse generator to cause successive stimulation pulses to be emitted by said pulse generator with a delay therebetween selected from the group consisting of an AV delay and a VV delay;
supplying said delay determining circuit with said hemodynamic parameter from said hemodynamic determining circuit and, in said delay determining circuit, iteratively adjusting said delay to optimize said delay with respect to said hemodynamic parameter; and
also supplying said delay determining circuit with said synchronicity measure from said hemodynamic parameter determining circuit and, in said delay determining circuit iteratively adjusting said delay to produce substantially synchronized movements of said valve plane at said first side and said second side during a cardiac cycle.

48. A method as claimed in claim 47 comprising placing said first valve plane electrode at a placement site selected from the group consisting of endocardially in the right atrium, endocardially in the left atrium, endocardially in the left ventricle, endocardially in the right ventricle, and epicardially, and placing said second valve plane electrode at a placement site selected from the group consisting of endocardially in the right atrium, endocardially in the left atrium, endocardially in the left ventricle, endocardially in the right ventricle, and epicardially.

49. A method as claimed in claim 42 comprising, said impedance measuring circuit, measuring, in each cardiac cycle, an extreme value of said impedance between said at least one first pair of electrodes and said impedance between said at least one second pair of electrodes, said extreme value being selected from the group consisting of a maximum and a minimum.

50. A method as claimed in claim 42 comprising in said impedance measuring circuit, determining an absolute value of a maximum derivative with respect to time for each impedance, in each cardiac cycle, between said at least one first pair of electrodes and between said at least one second pair of electrodes.

51. A method as claimed in claim 42 comprising, said impedance measuring circuit, measuring said impedance between each of said at least one first pair of electrodes and at least one second pair of electrodes during successive cardiac cycles.

52. A method as claimed in claim 42 comprising placing said at least one valve plane electrode at an endocardial placement site.

53. A method as claimed in claim 42 comprising placing said at least one valve plane electrode is an electrode at an epicardial placement site.

54. A computer-readable medium encoded with programming instructions, said medium being loadable into a control and sensing circuitry of an implantable medical device, having a pacing pulse generator that generates cardiac stimulation pacing pulses, and having a medical lead connected to said pulse generator and adapted for implantation in a subject to deliver the cardiac stimulating pacing pulses to cardiac tissue in vivo, said medical lead comprising at least a first pair of electrodes including at least one electrode adapted for location in an a atrium of the heart and at least one valve plane electrode adapted for location substantially at a level of a valve plane of the heart, and at least a second pair of electrodes comprising at least one electrode adapted for location in a ventricle of the heart and at least one electrode adapted for location at the level of said valve plane;
in an impedance measuring circuit connected to said medical lead, sense impedances reflecting movement of said valve plane by measuring an impedance between said first pair of electrodes and an impedance between said second pair of electrodes; and
in a hemodynamic parameter determining circuit supplied with said impedances measured by said impedance measuring circuit, to determine at least one hemodynamic parameter based on said impedances measured by said impedance measuring circuit, that represents mechanical functioning of the heart, said hemodynamic parameter determining circuit make said hemodynamic parameter available at an output thereof.
